(19) United States
(12) Patent Application Publication
(10) Pub. No.: US 2012/0129838 A1
(43) Pub. Date: May 24, 2012

(54) Composition for Preventing and Treating Alopecia Disorder Containing Morgalanthamine Compounds as an Active Ingredient

(75) Inventors: Young-Ho Kim, Daejeon (KR); Hee Kyoung Kang, Jeju-do (KR); Sang Cheol Kim, Jeju-do (KR); Jung Il Kang, Jeju-do (KR); Eun Sook Yoo, Jeju-do (KR); Ki Ho Kim, Chungcheongnam-do (KR); Young Heui Kim, Chungcheongnam-do (KR)

(73) Assignee: The Industry & Academic Cooperation in Chungnam National University, Daejeon (KR)

(21) Appl. No.: 13/265,820

(22) PCT Filed: Nov. 27, 2009
(86) PCT No.: PCT/KR09/07030

§ 371 Date: Feb. 8, 2012

(30) Foreign Application Priority Data
Apr. 24, 2009 (KR) 10-2009-0035814
Apr. 24, 2009 (KR) 10-2009-0035815

(51) Int. Cl. A61K 31/55; A61P 17/14; A61Q 5/08; A61Q 5/12; A61Q 5/00; A61Q 5/10; C07D 491/048; A61Q 5/02

(52) U.S. Cl. 514/215; 540/581

(57) ABSTRACT

Provided are a pharmaceutical composition, a cosmetic composition and health foods for preventing and treating alopecia disorder, which include a norgalanthamine compound as an active ingredient. Since the norgalanthamine compound has an effect on growth and proliferation of dermal papilla cells that play an important role in hair growth, the norgalanthamine compound may be used for the pharmaceutical composition, the cosmetic composition and health foods, which are useful in preventing and treating alopecia disorder.

### CROSS-REFERENCE TO RELATED APPLICATION

This application claims priority to and the benefit of Korean Patent Application No. 2009-0035814, filed Apr. 24, 2009, and Korean Patent Application No. 2009-0035815, filed Apr. 24, 2009, the disclosure of which is incorporated herein by reference in its entirety.

### BACKGROUND

1. Field of the Invention

The present invention relates to a pharmaceutical composition, a cosmetic composition and health foods for preventing and treating alopecia disorder, which include a norgalanthamine compound as an active ingredient.

2. Discussion of Related Art

In recent decades, there has been much research conducted to treat hair loss (alopecia). However, the causes of alopecia are still not precisely known. The causes of alopecia known up to the present include growth inhibition or malfunction of dermal papilla associated with the control of the hair cycle (Elliott et al. Differences in hair follicle dermal papilla volume are due to extracellular matrix volume and cell number: implications for the control of hair follicle size and androgen responses. J Invest Dermatol. 1990, Vol. 113, pages 873 to 877), abnormalization of the hair cycle by the action of male hormones, an abnormal change in the hair cycle caused by reduced blood flow to the scalp (Kaufman K D. Androgens and alopecia. Mol Cell Endocrinol. 2002, Vol. 198, pages 89 to 95), anti-cancer drugs (Botchkarev V A. Molecular mechanisms of chemotherapy-induced hair loss. J Investig Dermatol Symp Proc. 2003, Vol. 8, pages 72 to 75), mental stresses, physical stimuli, environmental pollution and the like (Botchkarev V A. Stress and the hair follicle: Exploring the connections. Am J Pathol. 2003, Vol. 162, pages 709 to 712).

Minoxidil and finasteride approved by the Food and Drug Administration (FDA) are known as drugs that facilitate hair growth. Minoxidil was first developed as a vasodilator to treat hypertension, but was developed as a hair restorer since it was reported to cause hirsutism as a side effect. The action mechanism of minoxidil for hair restoring effects was not clearly established, but an increase in supply of nutriments due to vasodilatation, potassium channel opening and inhibition of apoptosis of dermal papilla cells have been considered to induce the hair growth (Han et al. Effect of minoxodil on proliferation and apoptosis in dermal papilla cells of human hair follicle. J Dermatol Sci. 2004, Vol. 34, pages 91 to 98). Also, finasteride developed by Merck was developed as a therapeutic agent to treat prostatism since it inhibited the activities of an enzyme, 5α-reductase, which serves in the male hormone metabolism, but was developed as a hair restorer since it was reported to facilitate hair growth (Kaufman et al. Finasteride, a Type 2 5alpha-reductase inhibitor, in the treatment of men with androgenetic alopecia. Expert Opin Investig Drugs. 1999, Vol. 8, pages 403 to 415). There has been much research conducted by various research institutions to find many regulating factors associated with hair growth and alopecia mechanisms. In particular, it was reported that hair growth is controlled by signaling of various factors and their receptors, which are associated with the hair cycle including a growth phase (i.e., anagen), a transitional phase (i.e., catagen) and a resting phase (i.e., telogen). For example, growth factors such as EGF and EGFR (Botchkarev et al. Molecular control of epithelial-mesenchymal interactions during hair follicle cycling. J. Investig Dermatol Symp Proc. 2003, Vol. 8, pages 46 to 55), VEGF and VEGFR (Ozeki et al. In vivo promoted growth of mice hair follicles by the controlled release of growth factors. Biomaterials. 2003, Vol. 24, pages 2387 to 2394), HGF/SF and c-MET (Botchkarev et al. Molecular control of epithelial-mesenchymal interactions during hair follicle cycling. J Investig Dermatol Symp Proc. 2003, Vol. 8, pages 46 to 55), FGF family and FGFR (Jang J H. Stimulation of human hair growth by the recombinant human KGF-2. Biotechnol. Lett. 2005, Vol. 27, pages 749 to 752), IGF and IGF-IR (Kamiya et al. Hair follicle elongation in organ culture of skin from newborn and adult mice. Dermatol Sci. 1998, Vol. 17, pages 54 to 60), and TGF-β and TGF-βR (Hibino et al. Role of TGF-2 in the human hair cycle. J. Dermatol. Sci. 2004, Vol. 35, pages 9 to 18) were reported to affect the hair cycle under control of the activities of dermal papilla. Also, it was found that Wnt pathway and Bmp signaling are critical for proliferation or differentiation of hair follicle stem cells in a bulge region (Moore et al. Stem cells and their niches. Science. 2006, Vol. 311, pages 1880 to 1885).

The growth of hair takes place via a complicated mechanism including actions among various genes, growth factors and their receptors and systemic hormones. In particular, hair matrix cells composed of epithelial cells and dermal papilla cells composed of mesenchymal cells play a pivotal role in formation and growth of hair in hair follicles. Jahoda et al. (Jahoda et al. Induction of hair growth by implantation of cultured dermal papilla cells. Nature. 1984, Vol. 311, pages 560 to 562) reported that, when dermal papilla cells were removed from a rat vibrissa follicle, the growth of hair was temporarily stopped, and then continued again as cells moved to re-form dermal papilla cells from a dermal root sheath. Also, it was reported that when at most ⅓ of a lower portion of the hair follicle was removed, the growth of hair continued again as dermal papilla cells were formed from a dermal root sheath and hair matrix cells were formed from an epidermal root sheath, and, when at least ⅓ of the lower portion of the hair follicle was removed, the growth of hair did not continue. However, when at least ⅓ of the lower portion of the hair follicle was removed and separated dermal papilla cells were transplanted into the removed portion, the growth of hair continued. From these facts, it was seen that the dermal papilla cells play a very important role in the growth of hair.

Crinum asiaticum var. japonicum (hereinafter referred to as “C. asiaticum”) in Amaryllidaceae widely grows in Korea and Japan, and includes various kinds of phenanthridine alkaloids, triterpene alcohols and flavonoids (Min et al. Cytotoxic alkaloids and a flavan from the bulbs of Crinum asiaticum var. japonicum. Chem Pharm Bull (Tokyo). 2001, Vol. 49 (9), pages 1217 to 1219). In general, it has been known that alkaloids show various pharmacological effects such as an anti-viral effect (Gabrielsen et al. Antiviral (RNA) activity of selected Amaryllidaceae isoquinoline constituents and synthesis of related substances. J Nat Prod 1992, Vol. 55 (11), pages 1569 to 1581), an anti-malarial effect (Likhitwitayawuid et al. Cytotoxic and antimalarial alkaloids from the bulbs of Crinum amabile. J Nat Prod 1993, Vol. 56 (8), pages 1331 to 1338), a cytotoxic effect (Abdel-Halim et al. New crinine-type alkaloids with inhibitory effect on induction of inducible nitric oxide synthase from Crinum yemense. J Nat Prod 2004, Vol. 67 (7), pages 1119 to 1124), and an anti-inflammatory effect (Samud et al. Anti-inflammatory activity of Crinum asiaticum plant and its effect on bradykinin-induced contractions on isolated uterus. Immunopharmacology. 1999, Vol. 43 (2-3), pages 311 to 316). In addition, there are studies conducted on the effects of C. asiaticum on hair growth, and a patent application titled “A topical composition for preventing baldness and promoting hair growth” (Korean Patent Application No. 10-2008-27742, filed on Mar. 26, 2008) was filed in connection with the effects of C. asiaticum on the hair growth.

Therefore, the inventors have screened for components of C. asiaticum that shows a hair growth effect, conducted continuous research on prevention and treatment of alopecia disorder using a norgalanthamine compound which is one of the components of C. asiaticum, and found that the norgalanthamine compound shows effects on growth of vibrissa follicles in a rat and proliferation of dermal papilla cells that play an important role in the growth of hair. Accordingly, the present invention was completed based on these discoveries.

### SUMMARY OF THE INVENTION

The present invention is directed to providing a pharmaceutical composition, a cosmetic composition and health foods, which are effective in preventing and treating alopecia disorder.

In one aspect, the pharmaceutical composition for preventing and treating alopecia disorder may include a norgalanthamine compound represented by the following Formula I as an active ingredient.

In another aspect, the cosmetic composition for preventing and treating alopecia disorder may include a norgalanthamine compound represented by the following Formula I as an active ingredient.

In still another aspect, the health foods for preventing and treating alopecia disorder may include a norgalanthamine compound represented by the following Formula I as an active ingredient.

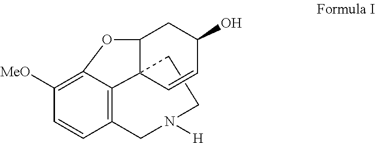

### BRIEF DESCRIPTION OF THE DRAWINGS

FIG. 1 is a diagram showing the effects of a norgalanthamine compound on growth of vibrissa follicles (% of control);

FIG. 2 is a diagram showing the effects of the norgalanthamine compound on proliferation of dermal papilla cells (% of control);

FIG. 3 shows a ¹³C-NMR spectrum (75 MHz, CD₃OD) of the norgalanthamine compound;

FIG. 4 shows a DEPT spectrum of the norgalanthamine compound;

FIG. 5 shows a ¹H-NMR spectrum (300 MHz, CD₃OD) of the norgalanthamine compound; and

FIG. 6 shows a FAB(+)-mass spectrum of the norgalanthamine compound.

### DETAILED DESCRIPTION OF EXEMPLARY EMBODIMENTS

Hereinafter, the present invention will be described in detail with respect to Examples according to the present invention and Comparative Examples not according to the present invention, but the scope of the present invention is not limited thereto.

In order to achieve the objects, the present invention provides a pharmaceutical composition for preventing and treating alopecia disorder, which includes a norgalanthamine compound represented by the following Formula I as an active ingredient.

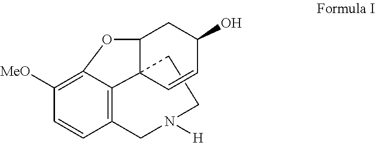

The norgalanthamine compound may, for example, be isolated from a C. asiaticum extract. Also, the norgalanthamine compound may be isolated from extracts of other plants or prepared using a conventional method such as chemical synthesis, but the present invention is not limited thereto.

According to one exemplary embodiment of the present invention, the alopecia disorder may be at least one disease selected from the group consisting of telogen effluvium, alopecia areata, anagen effluvium, alopecia traumatica, cicatricial alopecia and non-cicatricial alopecia, with telogen effluvium being preferred, but the present invention is not limited thereto.

According to another exemplary embodiment of the present invention, the pharmaceutical composition including the norgalanthamine compound of the present invention may include the norgalanthamine compound in a content of 0.1 to 50% by weight, for example, 1 to 50% by weight, or 1 to 40% by weight, based on the total weight of the composition. However, the contents of the components are not essentially limited thereto, but may be varied according to the condition of a patient, and the type and progress of a disease.

The norgalanthamine compound of the present invention may be safely used for a long period of time for preventive purposes since the compound has almost no toxicity or side effects.

The pharmaceutical composition of the present invention may further include a suitable component such as a carrier, an excipient and a diluent, which are usually used for preparation of a pharmaceutical composition.

The carrier, excipient and diluents which may be included in the pharmaceutical composition of the present invention may include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinyl pyrrolidone, water, methyl hydroxybenzoate, propyl hydroxybenzoate, talc, magnesium stearate and mineral oil. When the pharmaceutical composition is formulated, a formulation may be prepared using a commonly used diluent or excipient such as a filler, an extending agent, a binder, a wetting agent, a disintegrating agent and a surfactant. A solid formulation for oral administration includes a tablet, a pill, a powder, a granule, a capsule and the like. Such a solid formulation is prepared by mixing the norgalanthamine compound with at least one excipient such as, for example, starch, calcium carbonate, sucrose or lactose, or gelatin. Also, lubricants such as magnesium stearate and talc may be used in addition to the simple excipient. A liquid formulation for oral administration includes a suspension, an internal liquid preparation, an emulsion and syrup. In general, the liquid formulation may include various excipients, for example, a wetting agent, a sweetening agent, an aromatic and a preservative in addition to the commonly used simple diluent such as water and liquid paraffin. A formulation for parenteral administration includes a sterile aqueous solution, a non-aqueous solvent, a suspension, an emulsion, a lyophilized formulation, and a suppository. A vegetable oil such as propylene glycol, polyethylene glycol or olive oil, and an injectable ester such as such as ethyl oleate may be used as the non-aqueous solvent and the suspension. Witepsol, Macrogol, Tween 61, cacao butter, laurin butter or glycerogelatin may be used as a base of the suppository.

The pharmaceutical composition of the present invention may be formulated into an oral formulation such as powder, granule, tablet, capsule, suspension, emulsion, syrup or aerosol, an external preparation, a suppository and a sterile injectable solution using a conventional method.

A desirable dose of the pharmaceutical composition of the present invention may be varied according to the condition and weight of a patient, the severity of a disease, the type of a drug, and the route and duration of administration, or be suitably selected by those skilled in the art. In order to achieve the desirable effects, however, the norgalanthamine compound of the present invention may be administered daily at a dose of 0.5 g/kg to 5 g/kg, and preferably 1 g/kg to 3 g/kg. The administration may be performed once a day or in divided doses each day. Therefore the dosage is not intended to limit the scope of the present invention in any aspect.

According to one exemplary embodiment of the present invention, the pharmaceutical composition for preventing and treating alopecia disorder including the norgalanthamine compound of Formula I as an active ingredient may be a pharmaceutical composition for a skin external preparation. Here, the skin external preparation may be prepared into a cream, gel, patch, spray, ointment, plaster, lotion, liniment, pasta or cataplasma formulation, but the present invention is not limited thereto.

When the composition of the present invention is a pharmaceutical composition for a skin external preparation, a preferable dose of the norgalanthamine compound may be varied according to the condition and weight of a patient, the severity of a disease, the type of a drug, and the route and duration of administration, or be suitably selected by those skilled in the art. In order to achieve the desirable effects, however, the compound of the present invention may be administered daily at a dose of 0.0001 to 100 mg/kg, and preferably 0.001 to 10 mg/kg. The administration may be performed once a day or in divided doses each day. Therefore the dosage is not intended to limit the scope of the present invention in any aspect.

The composition of the present invention may be administered to a mammal such a rat, a mouse, a domestic animal and a human via various routes of administration. Any type of administration may be used including, for example, orally, rectally, or through intravenous, intramuscular or subcutaneous injection.

In addition, the present invention provides a cosmetic composition for preventing and treating alopecia disorder including the norgalanthamine compound of Formula I as an active ingredient. The cosmetic composition according to the present invention may be widely used in a hair care product that serves to prevent alopecia and enhance hair restoration.

According to one exemplary embodiment of the present invention, the cosmetic composition may be a formulation of a hair tonic, a hair conditioner, a hair essence, a hair lotion, a hair-nourishing lotion, a hair shampoo, a hair rinse, a hair treatment, a hair cream, a hair-nourishing cream, a hair moisture cream, a hair massage cream, a hair wax, a hair aerosol, a hair pack, a hair-nourishing pack, a hair soap, a hair cleansing foam, a hair oil, a hair drying preparation, a hair preserving and managing preparation, a hair dye, a hair waving preparation, a hair bleaching agent, a hair gel, a hair glaze, a hair dressinger, a hair lacquer, a hair moisturizer, a hair mousse or a hair spray, but the present invention is not particularly limited thereto.

The cosmetic composition for preventing alopecia and enhancing hair restoration according to the present invention may include the norgalanthamine compound in a content of 0.1 to 50% by weight, 1 to 50% by weight, or 1 to 40% by weight, based on the total weight of the composition. However, the content of the compound is not essentially limited thereto, but may be varied according to the condition of a subjected to be treated, and the progress of a disease.

A preferable dose of the norgalanthamine compound according to the present invention may be varied according to the condition and weight of a subject to be treated, the severity of a disease, the type of a drug, and the route and duration of administration, or be suitably selected by those skilled in the art. In order to achieve the desirable effects, however, the compound of the present invention may be administered daily at a dose of 0.0001 to 100 mg/kg, and preferably 0.001 to 10 mg/kg. The administration may be performed once a day or in divided doses each day. Therefore the dosage is not intended to limit the scope of the present invention in any aspect.

The cosmetic composition of the present invention may further include a composition selected from the group consisting of a water-soluble vitamin, an oil-soluble vitamin, a high-molecular peptide, a high-molecular polysaccharide, a sphingolipid and a seaweed extract.

The water-soluble vitamin may be used without limitation as long as it can be blended with cosmetics. Preferably, the water-soluble vitamin may include vitamin B1, vitamin B2, vitamin B6, pyridoxin, pyridoxine hydrochloride, vitamin B12, pantothenic acid, nicotinic acid, nicotinic acid amide, folic acid, vitamin C, or vitamin H. Also, salts (thiamine hydrochloride, ascorbate sodium, etc.) or derivatives (ascorbic acid-2-phosphate sodium, ascorbic acid-2-phosphate magnesium, etc.) of the above-described components may be included in the water-soluble vitamin which may be used in the present invention. The water-soluble vitamin may be obtained using a conventional method such as microbial transformation, purification from a culture solution of a microorganism, enzymatic or chemical synthesis.

The oil-soluble vitamin may be used without limitation as long as it can be blended with cosmetics. Preferably, the water-soluble vitamin may include vitamin A, carotin, vitamin D2, vitamin D3, vitamin E (d1-α tocopherol, d-α tocopherol, d-α tocopherol) and the like. Also, derivatives of the above-described components (ascorbic acid palmitate, ascorbic acid stearate, ascorbic acid dipalmitate, acetic acid d1-α tocopherol, nicotinic acid d1-α tocopherol vitamin E, DL-pantothenyl alcohol, D-pantothenyl alcohol, pantothenyl ethyl ether, etc.) are included in the oil-soluble vitamin which may be used in the present invention. The oil-soluble vitamin may be obtained using a conventional method such as microbial transformation, purification from a culture solution of a microorganism, enzymatic or chemical synthesis.

The high-molecular peptide may be used without limitation as long as it can be blended with cosmetics. Preferably, the high-molecular peptide may include collagen, hydrolyzed collagen, gelatin, elastin, hydrolyzed elastin, keratin, etc. The high-molecular peptide may be obtained in a purified form using a conventional method such as purification from a culture solution of a microorganism, enzymatic or chemical synthesis, or be purified from a conventional natural source such as thick skin from pigs or cattle, or fibroin from a silkworm.

The high-molecular polysaccharide may be used without limitation as long as it can be blended with cosmetics. Preferably, the high-molecular polysaccharide may include hydroxyethyl cellulose, xanthan gum, sodium hyaluronate, chondroitin sulphate or salts thereof (sodium salt, etc.). For example, chondroitin sulphate or a salt thereof may be generally purified from a mammal or a fish.

The sphingolipid may be used without limitation as long as it can be blended with cosmetics. Preferably, the sphingolipid may include ceramide, phytosphingosine, glycosphingolipid, etc. The sphingolipid may be generally purified from a mammal, a fish, a shellfish, yeast or a plant using a conventional method, or obtained using a conventional method such as chemical synthesis.

The seaweed extract may be used without limitation as long as it can be blended with cosmetics. Preferably, the seaweed extract may include a brown alga extract, a red alga extract, a green alga extract, etc. Also, carrageenan, alginic acid, sodium alginate, potassium alginate and the like purified from the seaweed extracts are included in the seaweed extract used in the present invention. The seaweed extract may be obtained from seaweed using a conventional method such as purification.

In addition to the essential components, the cosmetic composition of the present invention may include other components that may be blended into a conventional cosmetic composition, when necessary.

In addition to the above-described components, a blending component which may be added herein may include a fat component, a humectant, an emollient, a surfactant, organic and inorganic pigments, an organic powder, a UV absorber, an antiseptic, a bactericide, an antioxidant, an herbal extract, a pH regulating agent, an alcohol, a coloring matter, an aromatic, a blood flow stimulant, a cooling agent, an antiperspirant, purified water, etc.

The fat component may include ester-based fat, hydrocarbon-based fat, silicon-based fat, fluorine-based fat, animal fat, vegetable fat, etc.

The ester-based fat may include tri-2-ethylhexaneglyceryl, 2-ethylhexanecetyl, myristic acid isopropyl, myristic acid butyl, palmitic acid isopropyl, stearic acid ethyl, palmitic acid octyl, isostearic acid isocetyl, stearic acid butyl, linoleic acid ethyl, linoleic acid isopropyl, oleic acid ethyl, myristic acid isocetyl, myristic acid isostearyl, palmitic acid isostearyl, myristic acid octyldodecyl, isostearic acid isocetyl, sebacic acid diethyl, adipic acid diisopropyl, neopentanoic acid isoalkyl, tri(caprylic, capric acid)glyceryl, tri-2-ethylhexanetrimethylolpropane, triisostearic acid trimethylolpropane, tetra-2-ethylhexanepentaerythritol, caprylic acid cetyl, lauric acid decyl, lauric acid hexyl, myristic acid decyl, myristic acid myristyl, myristic acid cetyl, stearic acid stearyl, oleic acid decyl, ricinoleic acid cetyl, lauric acid isostearyl, myristic acid isotridecyl, palmitic acid isocetyl, stearic acid octyl, stearic acid isocetyl, oleic acid isodecyl, oleic acid octyldodecyl, linoleic acid octyldodecyl, isostearic acid isopropyl, 2-ethylhexanecetostearyl, 2-ethylhexanestearyl, isostearic acid hexyl, dioctanoic acid ethyleneglycol, dioleic acid ethyleneglycol, dicapric acid propylene glycol, di(caprylic, capric acid)propylene glycol, dicaprylic acid propylene glycol, dicapric acid neopentylglycol, dioctanoic acid neopentylglycol, tricaprylic acid glyceryl, triundecylic acid glyceryl, triisopalmitic acid glyceryl, triisostearic acid glyceryl, neopentanoic acid octyldodecyl, octanoic acid isostearyl, isononanoic acid octyl, neodecanoic acid hexyldecyl, neodecanoic acid octyldodecyl, isostearic acid isocetyl, isostearic acid isostearyl, isostearic acid octyldecyl, polyglycerineoleic acid ester, polyglycerineisostearic acid ester, citric acid triisocetyl, citric acid triisoalkyl, citric acid triisooctyl, lactic acid lauryl, lactic acid myristyl, lactic acid cetyl, lactic acid octyldecyl, citric acid triethyl, citric acid acetyltriethyl, citric acid acetyltributyl, citric acid trioctyl, malic acid diisostearyl, hydroxystearic acid 2-ethylhexyl, succinic acid di-2-ethylhexyl, adipic acid diisobutyl, sebacic acid diisopropyl, sebacic acid dioctyl, stearic acid cholesteryl, isostearic acid cholesteryl, hydroxystearic acid cholesteryl, oleic acid cholesteryl, oleic acid dihydrocholesteryl, isostearic acid phytosteryl, oleic acid phytosteryl, 12-stearoylhydroxystearic acid isocetyl, 12-stearoylhydroxystearic acid stearyl, 12-stearoylhydroxystearic acid isostearyl, etc.

The hydrocarbon-based fat may include squalene, liquid paraffin, α-olefin oligomer, isoparaffin, ceresin, paraffin, liquid isoparaffin, polybutene, microcrystalline wax, Vaseline, etc.

The silicon-based fat may include polymethylsilicon, methylphenylsilicon, methylcyclopolysiloxane, octamethylpolysiloxane, decamethylpolysiloxane, dodecamethylcyclosiloxane, a dimethylsiloxane/methylcetyloxysiloxane copolymer, a dimethylsiloxane/methylstearoxysiloxane copolymer, alkyl-modified silicon oil, amino-modified silicon oil, etc.

The fluorine-based fat may include perfluoropolyether, etc.

The animal or vegetable fat may include avocado oil, almond oil, olive oil, sesame oil, rice bran oil, safflower oil, soybean oil, corn oil, rapeseed oil, apricot oil, palm kernel oil, palm oil, castor oil, sunflower oil, grape seed oil, cottonseed oil, coconut oil, wheat germ oil, rice germ oil, shea butter, evening primrose oil, macadamia nut oil, meadowfoam oil, egg yolk oil, tallow, horse oil, mink oil, orange roughy oil, jojoba oil, candelilla wax, carnauba wax, liquid lanolin, hydrogenated castor oil, etc.

The humectant may include a water-soluble low-molecular humectant, a fat-soluble low-molecular humectant, a water-soluble polymer, a fat-soluble polymer, etc.

The water-soluble low-molecular humectant may include serine, glutamine, sorbitol, mannitol, pyrrolidone-sodium carboxylate, glycerine, propylene glycol, 1,3-butyleneglycol, ethyleneglycol, polyethyleneglycol B (degree of polymerization (n) of at least 2), polypropylene glycol (degree of polymerization (n) of at least 2), polyglycerine B (degree of polymerization (n) of at least 2), lactic acid, lactate, etc.

The fat-soluble low-molecular humectant may include cholesterol, cholesterolester, etc.

The water-soluble polymer may include carboxyvinyl polymer, polyaspartate, tragacanth, xanthan gum, methyl cellulose, hydroxymethylcellulose, hydroxyethyl cellulose, hydroxypropyl cellulose, carboxymethyl cellulose, water-soluble chitin, chitonic acid, dextrin, etc.

The fat-soluble polymer may include a polyvinylpyrrolidone/eicosene copolymer, a polyvinylpyrrolidone/hexadecene copolymer, nitrocellulose, dextrin fatty acid ester, polymer silicon, etc.

The emollient may include long-chain acyl glutamic acid cholesteryl ester, hydroxystearic acid cholesteryl, 12-hydroxystearic acid, stearic acid, rosin acid, lanolin fatty acid cholesteryl ester, etc.

The surfactant may include a non-ionic surfactant, an anionic surfactant, a cationic surfactant, an amphoteric surfactant, etc.

The non-ionic surfactant may include self-emulsified monostearic acid glycerine, propylene glycol fatty acid ester, glycerine fatty acid ester, polyglycerine fatty acid ester, sorbitan fatty acid ester, polyoxyethylene (POE) sorbitan fatty acid ester, POE sorbite fatty acid ester, POE glycerine fatty acid ester, POE alkylether, POE fatty acid ester, POE hydrogenated caster oil, POE castor oil, a POE/polyoxypropylene (POP) copolymer, POE/POP alkylether, polyether-modified silicon, lauric acid alkanol amide, alkylamine oxide, hydrogenated soybean phospholipid, etc.

The anionic surfactant may include fatty acid soap, α-acylsulfonate, alkylsulfonate, alkylarylsulfonate, alkylnaphthalenesulfonate, alkylsulfate, POE alkylethersulfate, alkylamidesulfate, alkylphosphate, POE alkylphosphate, alkylamidephosphate, alkyloylalkyltaurate, N-acylamino acid salt, POE alkylethercarboxylate, alkylsulfosuccinate, sodium alkylsulfoacetate, acylated hydrolyzed collagen peptide salt, perfluoroalkyl phosphate ester, etc.

The cationic surfactant may include alkyltrimethyl ammonium chloride, stearyltrimethyl ammonium chloride, stearyltrimethylammonium bromide, cetostearyltrimethyl ammonium chloride, distearyldimethyl ammonium chloride, stearyldimethylbenzyl ammonium chloride, behenyltrimethyl ammonium bromide, benzalkonium chloride, diethylaminoethylamide stearate, dimethylaminopropylamide stearate, quaternary ammonium derivatives of lanolin, etc.

The amphoteric surfactant may include carboxybetaine-type, amide betaine-type, sulfobetaine-type, hydroxyl sulfobetaine-type, amide sulfobetaine-type, phosphobetaine-type, aminocarboxylate-type, imidazoline derivative-type, amideamine-type amphoteric surfactants, etc.

The organic and inorganic pigment may include an inorganic pigment such as silicic acid, silica, magnesium silicate, talc, sericite, mica, kaolin, rouge, clay, bentonite, titan-coated mica, bismuth oxychloride, zirconium oxide, magnesium oxide, zinc oxide, titanium oxide, aluminium oxide, calcium sulfate, barium sulfate, magnesium sulfate, calcium carbonate, magnesium carbonate, iron oxide, ultramarine, chromium oxide, chromium hydroxide, calamine and a complex thereof; an organic pigment such as polyamide, polyester, polypropylene, polystyrene, polyurethane, vinyl resin, urea resin, phenol resin, fluorine resin, silica resin, acrylic resin, melamine resin, epoxy resin, polycarbonate resin, a divinylbenzene/styrene copolymer, silk powder, cellulose, CI Pigment Yellow, or CI Pigment Orange; and a complex pigment of the inorganic pigment and the organic pigment, etc.

The organic powder may include a metallic soap such as calcium stearate; a metal alkylphosphate such as zinc sodium cetylate, zinc laurylate or calcium laurylate; a polyvalent acylamino acid metal salt such as N-lauroyl-β-alanine calcium, N-lauroyl-β-alanine zinc or N-lauroyl glycine calcium; a polyvalent amide sulfonic acid metal salt such as N-lauroyl-taurine calcium or N-palmitoyl-taurine calcium; a N-acyl basic amino acid such as N-ε-lauroyl-L-lysine, N-ε-palmitoyllysine, N-α-palmitoyl ornithine, N-α-lauroylarginine, or N-α-hydrogenated tallow fatty acid acyl arginine; an N-acylpolypeptide such as N-lauroylglycylglycine; an α-amino fatty acid such as α-amino caprylic acid, or α-amino lauric acid; polyethylene, polypropylene, nylon, polymethylmethacrylate, polystyrene, divinylbenzene/styrene copolymer, tetrafluoroethylene, etc.

The UV absorber may include para-amino benzoic acid, ethyl-para-benzoate, amyl-para-aminobenzoate, octyl-para-aminobenzoate, salicylic acid ethylene glycol, henyl salicylate, octyl salicylate, benzyl salicylate, butylphenyl salicylate, homomentyl salicylate, benzyl cinnamate, 2-ethoxyethyl para-methoxy cinnamate, octyl para-methoxy cinnamate, mono-2-ethyl hexane glyceryl di-para-methoxy cinnamate, isopropyl para-methoxy cinnamate, a diisopropyl/diisopropyl cinnamic acid ester mixture, urocanic acid, ethyl urocanate, hydroxy methoxybenzophenone, hydroxyl methoxybenzophenone sulfonic acid and slats thereof, dihydroxy methoxybenzophenone, sodium dihydroxy methoxybenzophenone disulfonate, dihydroxy benzophenone, tetrahydroxy benzophenone, 4-tert-butyl-4′-methoxy dibenzoylmethane, 2,4,6-trianilino-p-(carbo-2′-ethylhexyl-1′-oxy)-1,3,5-triazine, 2-(2-hydroxy-5-methylphenyl)benzotriazole, etc.

The bactericide may include hinokitiol, triclosan, trichlorohydroxydiphenyl ether, chlorhexidine gluconate, phenoxy ethanol, resorcin, isopropylmethylphenol, azulene, salicylic acid, zinc pyrithione, benzalkonium chloride, photosensitizer 301, sodium mononitroguaiacol, undecylenic acid, etc.

The antioxidant may include butylhydroxy anisole, propyl gallate, erythorbic acid, etc.

The pH regulating agent may include citric acid, sodium citrate, malic acid, sodium malate, fumaric acid, sodium fumarate, succinic acid, sodium succinate, sodium hydroxide, disodium hydrogen phosphate, etc.

The alcohol may include a higher alcohol such as cetyl alcohol.

In addition, a blending component which may be added herein is not limited to the above-described components, and any component may be blended in such a range that the objects and effects of the present invention are not hindered. That is, the component may be preferably blended in a content of 0.01 to 5% by weight, and more preferably a content of 0.01 to 3% by weight, based on the total weight of the composition.

The cosmetic composition of the present invention may be prepared in the form of solution, emulsion or viscous mixture.

In addition to the compound, the components included in the cosmetic composition of the present invention may further include components generally used for a cosmetic composition as active ingredients. For example, the cosmetic composition includes a conventional adjuvant and carrier such as a stabilizing agent, a solubilizing agent, a vitamin, a pigment and an aromatic.

The cosmetic composition of the present invention may be prepared into any formulation which is generally prepared in the art, and examples of the formulation may include a milky lotion, a cream, a face lotion, a pack, a foundation lotion, a lotion, an essence, a hair care composition, etc.

When a formulation of the present invention is in the form of a paste, cream or gel, an animal fiber, a vegetable fiber, a wax, paraffin, a starch, tragant, a cellulose derivative, polyethylene glycol, silicon, bentonite, silica, talc or zinc oxide may be used as a carrier component.

When the formulation of the present invention is in the form of a powder or spray, lactose, talc, silica, aluminum hydroxide, calcium silicate or polyamide powder may be used as the carrier component. Particularly when the formulation of the present invention is in the form of spray, the formulation may further include a propellent such as chlorofluorohydrocarbon, propane/butane or dimethylether.

When the formulation of the present invention is in the form of a solution or emulsion, a solvent, a solvating agent or an emulsifying agent may be used as the carrier component. For example, the carrier component may include water, ethanol, isopropanol, ethyl carbonate, ethyl acetate, benzyl alcohol, benzyl benzoate, propylene glycol, 1,3-butylglycol oil, aliphatic glycerol ester, polyethylene glycol, or sorbitan fatty acid ester.

When the formulation of the present invention is in the form of a suspension, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum methahydroxide, bentonite, agar or tragant may be used as the carrier component.

When the formulation of the present invention is in the form of surfactant-containing cleansing, aliphatic alcohol sulfate, aliphatic alcohol ether sulfate, sulfosuccinic acid monoester, isethionate, imidazolium derivative, methyltaurate, sarcosinate, fatty acid amide ether sulfate, alkylamido betaine, aliphatic alcohol, fatty acid glyceride, fatty acid diethanolamide, a vegetable oil, a linolin derivative or ethoxylated glycerol fatty acid ester may be used as the carrier component.

Also, the present invention provides a health food for preventing and treating alopecia disorder including the norgalanthamine compound of Formula I as an active ingredient.

The composition including the norgalanthamine compound according to the present invention may be widely used in a drug, food and beverage to prevent and treat alopecia disorder. The food to which the compound of the present invention can be added may, for example, include various foods, beverages, gums, teas, vitamin complexes, and health food supplements, but the present invention is not limited thereto. For example, the food may be used in the form of a powder, granule, tablet, capsule or beverage.

The compound of the present invention may be added to a food or a beverage for the purpose of preventing and treating alopecia disorder. In the case of the health food composition, the compound in the food or beverage may be generally added in a content of 1 to 5% by weight, based on the total weight of the food. Also, in the case of the health beverage composition, the compound may be added at a ratio of 0.02 to 10 g, and preferably 0.3 to 1 g, based on 100 ml of the composition.

The health beverage composition of the present invention includes the compound as an essential component at an indicated ratio, but may include other liquid components without particular content limitation. Like a conventional beverage, the health beverage composition may include an additional component such as various flavoring agents or natural carbohydrates. Examples of the above-described natural carbohydrates include a monosaccharide such as glucose, a disaccharide such as fructose, a polysaccharide such as maltose or sucrose, a conventional sugar such as dextrin or cyclodextrin, and a sugar alcohol such as xylitol, sorbitol or erythritol. In addition to the above-described components, a natural flavoring agent (thaumatin, a stavia extract (i.e., rebaudioside A, glycyrrhizin, etc.)) and a synthetic flavoring agent (saccharin, aspartame, etc.) may be advantageously used as the flavoring agent. The natural carbohydrate may be generally present in a content of approximately 1 to 20 g, and preferably approximately 5 to 12 g, based on 100 ml of the composition of the present invention.

In addition to the above-described components, the composition of the present invention may include a variety of nutrients, a vitamin, a mineral (an electrolyte), a flavoring agent such as a synthetic flavoring agent or a natural flavoring agent, a coloring agent and an improving agent (cheese, chocolate, etc.), pectic acid and salts thereof, alginic acid and salts thereof, organic acid, a protective colloidal thickener, a pH regulating agent, a stabilizing agent, an antiseptic, glycerine, alcohol, and a carbonating agent used in a carbonated beverage. In addition, the compositions of the present invention may include a pulp for preparation of a natural fruit and vegetable juice. These components may be used alone or in combination. A content of such an additive is inconsequential, but generally selected from a range of 0 to approximately 20 parts by weight, based on 100 parts by weight of the composition of the present invention.

Hereinafter, the present invention will be described in detail with reference to Reference Examples, Examples and Experimental Examples.

However, it should be understood that the following Reference Examples, Examples and Experimental Examples proposed herein are merely described for the purpose of illustration only, and not intended to limit the scope of the invention.

### Example 1

### Separation and Purification of Norgalanthamine

An aerial part of C. asiaticum growing wild in Jeju-do (Korea) was collected and dried in the shade to yield a sample. 1.5 kg of the sample was cut into pieces, extracted three times from 25,000 ml of methanol, and concentrated to give 500 g of a methanol extract. The methanol extract was sequentially fractionated with 1,000 ml of hexane, dichloromethane, ethyl acetate and butanol. Then, a butanol fraction was subjected to silica gel column chromatography using chloroform-methanol-water (13:7:2(v/v/v), a lower layer) as a mobile phase to obtain 9 subtractions (“Fr.1” to “Fr.9”). Subfraction Fr.4 was suspended in water and dichloromethane according to the above-mentioned method, and sequentially treated with an acid and a base to obtain an alkaloid fraction. The alkaloid fraction was also subjected to silica gel column chromatography using acetone-water (2:1(v/v)) as a mobile phase to obtain a crude norgalanthamine fraction. Then, the crude norgalanthamine fraction was re-crystallized from acetone to obtain 160 mg of a white acicular crystal, norgalanthamine.

White Acicular Crystal

Melting Point: 179-181° C.

[ ]_{D}²⁰ (MeOH, c 0.29): −65.5°

UVₘₐₓ: 278, 288 nm

FAB-MS m/z: 274 [M+H]⁺

¹H-NMR (300 MHz, CD₃OD): 2.52 (H, d, J=15.9 Hz, H-1), 2.18 (H, d, 15.9 Hz, H-1), 4.21 (H, m, H-2), 6.02 (H, d, J=10.3 Hz, H-3), 6.16 (H, d, J=10.3 Hz, H-4), 2.08 (2H, m, H-6), 3.65 (H, d, J=15.0 Hz, H-7), 3.63 (H, d, J=15.0 Hz, H-7), 4.34 (H, d, J=15.0 Hz, H-9), 4.54 (H, d, J=15.0 Hz, H-9), 6.83 (H, d, J=8.3 Hz, H-11), 6.86 (H, d, J=8.3 Hz, H-12), 4.63 (H, m, H-16), 3.83 (3H, s, H—OCH₃)

¹³C-NMR (75 MHz, CD₃OD): 31.40 (C-1), 62.08 (C-2), 129.99 (C-3), 127.00 (C-4), 49.28 (C-5), 35.94 (C-6), 46.62 (C-7), 51.74 (C-9), 123.04 (C-10), 123.55 (C-11), 113.60 (C-12), 148.43 (C-13), 147.04 (C-14), 134.20 (C-15), 88.57 (C-16), 56.66 (OCH₃)

### Reference Example 1

### Statistical Analysis

All measured data was represented by average standard deviation, and a statistical significance test was performed using a Student's t test. Here, when a p-value was equal to or less than 0.05, it was proven to be significant. The statistical analysis was performed using SPSS 12.0K for Windows (Release 12.0.1. SPSS Inc. USA).

### Experimental Example 1

### Measurement of Effect on Vibrissa Follicle Growth

1-1. Isolation and Culture of Rat Vibrissa Follicles

In order to measure an effect of the norgalanthamine compound prepared in Experimental Example 1 on hair growth, the following experiment was performed using a method described in the publication (Philpott M P, et al., Cyclical changes in rat vibrissa follicles maintained in vitro. J Invest Dermatol. 115, pp. 1152-1155, 2000),

A 3-week-old male Wistar rat (Japan SLC, Hamamatsu, Japan) was purchased from Central Lab. Animal Inc. (Korea), anesthetized with ethyl ether, and killed by cervical vertebral dislocation. The left and right mystacial pads of the rat were extracted and put into an E/P buffer ((EBSS, Earle's balanced salts solution, Sigma Mo., USA)+(PBS, phosphate-buffered saline, Sigma Mo., USA)) supplemented with 100 units/ml penicillin and 100 μg/ml streptomycin (Gibco Inc, NY, USA). Vibrissa follicles were carefully isolated under a steroscopic dissecting microscope. Until the hair follicles were completely isolated, the hair follicles were put into a Petri dish containing an E/P buffer, and incubated at 37° C. for 1 hour in a 5% CO₂ pyrostat. 500 μl of William E medium (Gibco Inc, NY, USA) containing 2 mM. L-glutamine (Gibco Inc, NY, USA), 10 μg/ml insulin (Sigma Mo., USA), 50 nM hydrocortisone (Sigma Mo., USA) and 100 units/ml penicillin-100 μg/ml streptomycin was put into each well of a 24-well plate, and one hair follicle was added to each well, and incubated at 37° C. hour in a 5% CO₂ pyrostat. 10 to 12 hair follicles were used in each experimental group, and a medium was changed with a fresh medium every 3 days during the incubation. The hair follicles were treated with 0.01 and 0.1 uM concentrations of the norgalanthamine compound of Experimental Example 1, and treated with a 1 μM concentration of minoxidil sulfate (M.S., Sigma, USA) as a positive control.

1-2. Measurement of Growth of Rat Vibrissa Follicles

Photographs of shapes of vibrissa follicles were taken using a microscope (Olympus, Japan). Lengths of hair follicles were measured at time points of 0, 7, 14 and 21 days using an image analyzer (DP controller; Olympus, Japan). An average variation in lengths of the hair follicles was calculated, and a growth level was measured by comparing the average variation with an average length of the control.

1-3. Experimental Results

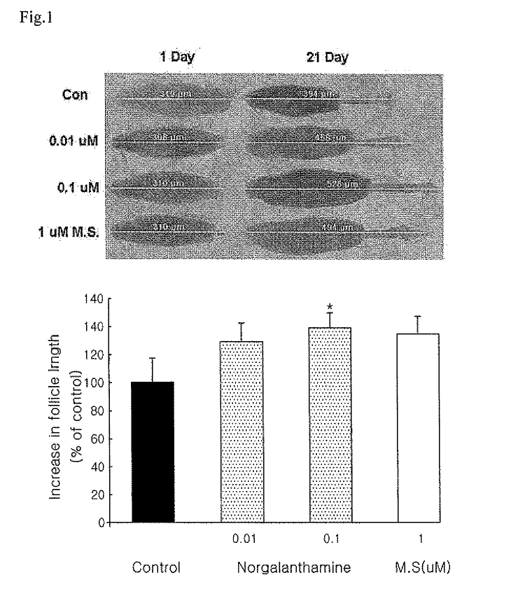

According to the experimental results, the effects of the norgalanthamine compound on the hair growth were confirmed by isolating vibrissa follicles from a 3-week-old rat in a growth phase, incubating the vibrissa follicles for 3 weeks and measuring lengths of the hair follicles. As shown in FIG. 1, a difference (%) in length of hair follicles between the norgalanthamine-treated group and the control was examined. As a result, when the hair follicles were treated with 0.01 and 0.1 uM concentrations of norgalanthamine, respectively, the growth of hair fiber at a time point of 21 days was significantly increased to 128.7±13.8% and 139.2±10.3% (P>0.05), compared to that of the control (100%), and the treatment with 1 uM minoxidil sulfate (M.S.) as a positive control increased the growth of hair fiber to 135.03±38.8%.

### Experimental Example 2

### Measurement of Effects on Proliferation of Dermal Papilla Cells

In order to confirm whether the norgalanthamine compound of Experimental Example 1 showed an effect on proliferation of dermal papilla cells, which play a very important role in the growth of hair, a rat vibrissa immortalized dermal papilla cell line was tested by an MTT assay using a method described in the publication (Han J H, et al., Effect of minoxodil on proliferation and apoptosis in dermal papilla cells of human hair follicle., J Dermatol Sci, 34, pp. 91-98, 2004),

A rat vibrissa immortalized dermal papilla cell line obtained by immortalizing dermal papilla cells isolated from rat whiskers (Filsell W, et al., Transfection of rat dermal papilla cells with a gene encoding a temperature-sensitive polyomavirus large T antigen generates cell lines a differentiated phenotype., J. Cell Sci, 107, pp. 1761-1772, 1994) was incubated at 37° C. in a DMEM (Hyclone Inc, USA) medium containing 100 units/ml μg/ml streptomycin (Gibco Inc, NY, USA) and 10% heat-inactivated fetal bovine serum (FBS, Gibco Inc, NY, USA) using a 5% CO₂ pyrostat, and subcultured every 3 days.

The cultured dermal papilla cells (1.0×10⁴ cells/ml) were put into each well of a 96-well plate, and incubated for 24 hours. Then, a used culture solution was changed with a serum-free DMEM medium, and cultured again for 24 hours. Thereafter, the cultured solution was treated with 0.01 and 0.1 μM concentrations of the norgalanthamine compound of Experimental Example 1, and a 1 μM concentration of a positive control, minoxidil sulfate (M.S., Sigma, Mo., USA), respectively. The resulting mixture was incubated for 4 days, 50 μl of MTT (Sigma, Mo., USA) was added thereto, and the mixture was reacted for 4 hours. A supernatant was removed, and a pellet was dissolved in 200 μl of DMSO, and then measured for absorbance at 540 nm using a microplate reader (Amersham Pharmacia Biotech, NY, USA). An average absorbance value of each sample group was calculated, and a level of the hair proliferation was examined by comparing the average absorbance value with an absorbance value of the control.

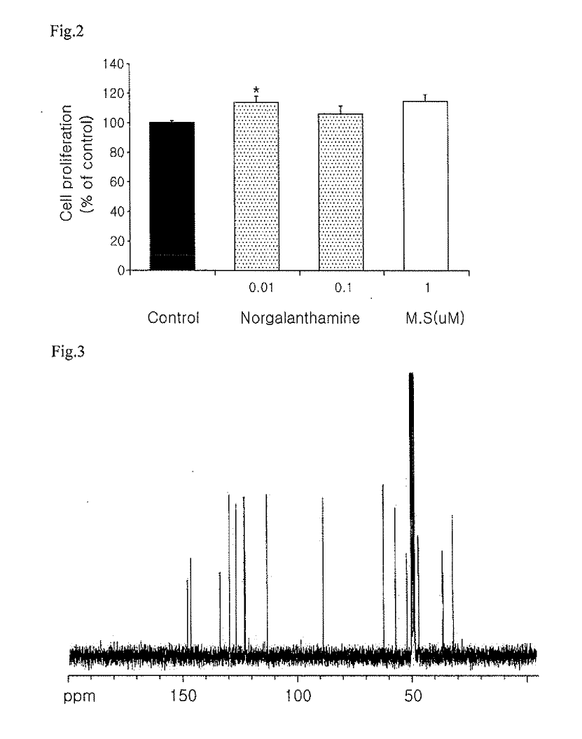

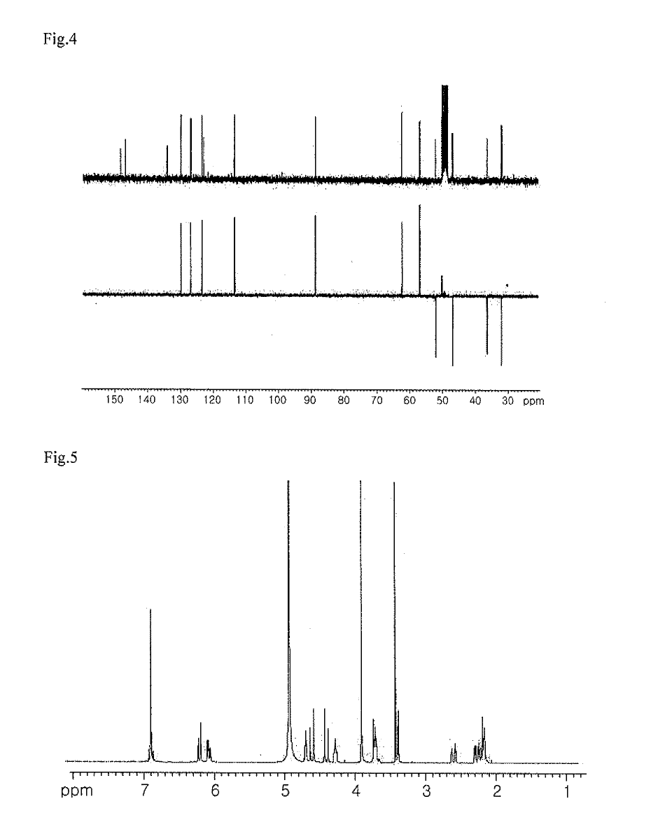

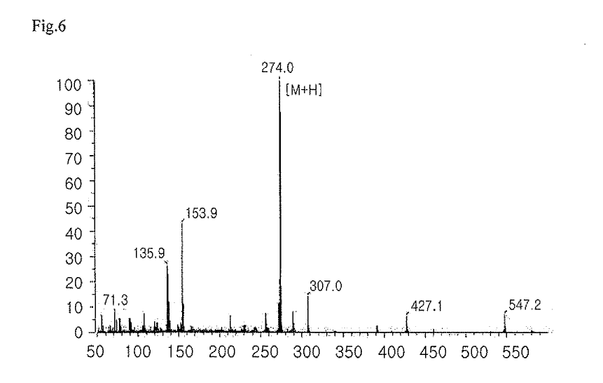

According to the experimental results, as shown in FIG. 2, it was confirmed that, when the hair follicles were treated with 0.01 and 0.1 μM concentrations of the norgalanthamine compound of the present invention, the growth of hair was significantly increased to 114.0±4.3% (P>0.05) and 106.2±5.6%, compared to the proliferation of the control (100%), respectively, and that the effect of the norgalanthamine compound on proliferation of dermal papilla cells was increased to a level similar to the effect of minoxidil sulfate (M.S.), which was widely known as a representative drug promoting hair growth, on the proliferation of dermal papilla cells (114.9±4.5%).

In conclusion, the norgalanthamine compound, which was one of the components of C. asiaticum, was considered to serve to induce or maintain the growth of hair follicles by facilitating the growth and proliferation of dermal papilla cells which play a very important role in hair growth. Therefore, the norgalanthamine compound may be effectively used to prevent and treat alopecia.

Hereinafter, Preparation Examples of the composition including the compound according to the present invention will be described. However, it should be understood that the description proposed herein is merely described for the purpose of illustration only, and not intended to limit the scope of the invention.

### Preparation Example 1

### Preparation of Pharmaceutical Composition

### Preparation Example 1-1

### Preparation of Powder

| | | |
|---|---|---|
| Norgalanthamine Compound | 20 | mg |
| Lactose | 100 | mg |
| Tale | 10 | mg |

The components were mixed, and an airtight bag was then filled with the mixed components to prepare a powder.

### Preparation Example 1-2

### Preparation of Tablet

| | | |
|---|---|---|
| Norgalanthamine Compound | 10 | mg |
| Corn Starch | 100 | mg |
| Lactose | 100 | mg |
| Magnesium Stearate | 2 | mg |

The components were mixed, and then compressed into a tablet using a conventional method of preparing a tablet.

### Preparation Example 1-3

### Preparation of Capsule

| | | |
|---|---|---|
| Norgalanthamine Compound | 10 | mg |
| Crystalline Cellulose | 3 | mg |
| Lactose | 14.8 | mg |
| Magnesium Stearate | 0.2 | mg |

Using a conventional method of preparing a capsule, the components were mixed, and a gelatin capsule was filled with the mixed components to prepare a capsule.

### Preparation Example 1-4

### Preparation of Injectable Solution

| | | |
|---|---|---|
| Norgalanthamine Compound | 10 | mg |
| Mannitol | 180 | mg |
| Injectable Sterile Distilled Water | 2,974 | mg |
| Na₂HPO₄, 12H₂O | 26 | mg |

The above-described components were mixed, and 2 ml of the resulting mixture was put into each ampule according to a conventional method of preparing an injectable solution.

### Preparation Example 1-5

### Preparation of Solution

| | | |
|---|---|---|
| Norgalanthamine Compound | 20 | mg |
| Isomerized Sugar | 10 | g |
| Mannitol | 5 | g |

| | |
|---|---|
| Purified Water | Proper Amount |

A solution was prepared using a conventional method of preparing a solution by dissolving the above-described components in purified water, adding a lemon flavor thereto, mixing all the components, adding purified water to the resulting mixture so that a final amount of the mixture was adjusted to 100 ml, putting the mixture into a brown vial, and sterilizing the mixture.

### Preparation Example 2

### Preparation of Cosmetic Composition

### Preparation Example 2-1

### Preparation of Hair Tonic

| | |
|---|---|
| Resorcinol | 0.1% |
| Menthol | 0.05% |
| Panthenol | 0.2% |
| Salicylic Acid | 0.1% |
| Tocopheryl acetate | 0.1% |
| Pyridoxine Hydrochloride | 0.1% |
| Castor Oil | 5.0% |
| Norgalanthamine Compound | 10.0% |
| Coloring Matter | Proper Amount |
| Aromatic | Proper Amount |
| Ethanol | Proper Amount |
| Purified Water | Balance |
| Total | 100.0% |

### Preparation Example 2-2

### Preparation of Hair Conditioner

| | |
|---|---|
| Cetanol | 3.5% |
| Self-Emulsified Monostearic Acid Glycerine | 1.5% |
| Propylene Glycol | 2.5% |
| Stearylmethylbenzyl Ammonium Chloride (25%) | 7.0% |
| Methyl Para-Oxybenzoate | 0.3% |
| Norgalanthamine Compound | 2.5% |
| Coloring Matter | Proper Amount |
| Aromatic | Proper Amount |
| Purified Water | Balance |
| Total | 100.0% |

### Preparation Example 2-3

### Preparation of Hair Lotion

| | |
|---|---|
| Resorcinol | 2.0% |
| Menthol | 2.0% |
| Panthenol | 0.5% |
| Piroctone Olamine | 0.1% |
| Norgalanthamine Compound | 5.0% |
| Aromatic/Coloring Matter | 0.5% |
| Purified Water | Balance |
| Total | 100.0% |

### Preparation Example 2-4

### Preparation of Hair Soap

| | |
|---|---|
| Norgalanthamine Compound | 0.1% |
| Titanium Dioxide | 0.2% |
| Polyethylene Glycol | 0.8% |
| Glycerine | 0.5% |
| Ethylene Diamine Tetraacetic Acid | 0.05% |
| Sodium | 1.0% |
| Coloring Matter | Proper Amount |
| Soap Flavor | Proper Amount |
| Cosmetic Soap Base (Moisture: 13% by weight) | Balance |
| Total | 100.0% |

### Preparation Example 2-5

### Hydrophilic Ointment

| | | Content | Content |
|---|---|---|---|
| Nos. | Materials | (% by weight) | (% by weight) |
| 1 | Norgalanthamine Compound | 0.5 | — |
| 2 | White Vaseline | 250 | 250 |
| 3 | Stearyl Alcohol | 220 | 220 |
| 4 | Ethyl (or Methyle) p-Oxybenzoate | 0.25 | 0.25 |
| 5 | Propylene Glycol | 120 | 120 |
| 6 | Sodium Lauryl Sulfate | 15 | 15 |
| 7 | Propyl p-Oxybenzoate | 0.15 | 0.15 |

### Preparation Example 3

### Preparation of Health Foods

### Preparation Example 3-1

### Preparation of Health Food

| Norgalanthamine Compound | 1000 | mg |
|---|---|---|
| Vitamin Blend | Proper Amount | |
| Vitamin A Acetate | 70 | μg |
| Vitamin E | 1.0 | mg |
| Vitamin B1 | 0.13 | mg |
| Vitamin B2 | 0.15 | mg |
| vitamin B6 | 0.5 | mg |
| Vitamin B12 | 0.2 | μg |
| Vitamin C | 10 | mg |
| Biotin | 10 | μg |
| Nicotinic Acid Amide | 1.7 | mg |
| Folic Acid | 50 | μg |
| Calcium Pantothenate | 0.5 | mg |
| Mineral Mixture | Proper Amount | |
| Ferrous Sulfate | 1.75 | mg |
| Zinc Oxide | 0.82 | mg |
| Magnesium Carbonate | 25.3 | mg |
| Potassium Phosphate Monobasic | 15 | mg |
| Calcium Phosphate Dibasic | 55 | mg |
| Potassium Citrate | 90 | mg |
| Calcium Carbonate | 100 | mg |
| Magnesium Chloride | 24.8 | mg |

The composition ratios of the vitamins and the mineral mixture were obtained by mixing relatively suitable components for a health food in preferable Experimental Examples, but may be optionally varied. Then, the above-described components were mixed to prepare a granule according to a conventional method of preparing a health food, and may be used for preparation of a health food composition according to a conventional method.

### Preparation Example 3-2

### Preparation of Health Beverage

| | | |
|---|---|---|
| Norgalanthamine Compound | 1000 | mg |
| Citric Acid | 1000 | mg |
| Oligosaccharide | 100 | g |
| Japanese Apricot Extract | 2 | g |
| Taurine | 1 | g |
| Purified Water | to 900 | ml |

The above-described components were mixed according to a conventional method of preparing a health beverage, and heated at 85° C. for approximately 1 hour while stirring. The resulting mixture was filtered and put into a 2-L sterile container. Thereafter, the sterile container was sealed, sterilized and refrigerated. Then, the mixture was used to prepare a health beverage composition of the present invention.

The composition ratios of the components were obtained by mixing relatively suitable components for a health beverage in preferable Experimental Examples, but may be optionally varied according to classes and countries on demand, and regional and national preferences such as usage.

Since the norgalanthamine compound of the present invention has an effect on the growth and proliferation of dermal papilla cells which play an important role in the growth, the norgalanthamine compound may be used in a pharmaceutical composition, a cosmetic composition and health foods which are useful in preventing and treating alopecia disorder.

While the invention has been shown and described with reference to predetermined exemplary embodiments thereof, it will be understood by those skilled in the art that various changes in form and details may be made therein without departing from the scope of the invention as defined by the appended claims.

What is claimed is:

1. A pharmaceutical composition for preventing and treating alopecia disorder, comprising a norgalanthamine compound represented by the following Formula I as an active ingredient.

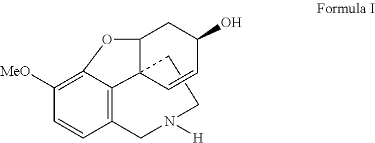

2. The pharmaceutical composition according to claim 1, wherein the alopecia disorder is telogen effluvium, alopecia areata, anagen effluvium, alopecia traumatica, cicatricial alopecia or non-cicatricial alopecia.

3. The pharmaceutical composition according to claim 1, wherein the compound is included at a content of 0.1 to 50% by weight, based on the total weight of the pharmaceutical composition.

4. The pharmaceutical composition according to claim 1, wherein the pharmaceutical composition is a skin external preparation.

5. The pharmaceutical composition according to claim 4, wherein the norgalanthamine compound is administered in a daily dose of 0.0001 to 100 mg/kg.

6. The pharmaceutical composition according to claim 4, wherein the skin external preparation is a cream, gel, patch, spray, ointment, plaster, lotion, liniment, pasta or cataplasma formulation.

7. A cosmetic composition for preventing and treating alopecia disorder, comprising a norgalanthamine compound of Formula I defined in claim 1 as an active ingredient.

8. The cosmetic composition according to claim 7, wherein the cosmetic composition is a formulation of a hair tonic, a hair conditioner, a hair essence, a hair lotion, a hair-nourishing lotion, a hair shampoo, a hair rinse, a hair treatment, a hair cream, a hair-nourishing cream, a hair moisture cream, a hair massage cream, a hair wax, a hair aerosol, a hair pack, a hair-nourishing pack, a hair soap, a hair cleansing foam, a hair oil, a hair drying preparation, a hair preserving and managing preparation, a hair dye, a hair waving preparation, a hair bleaching agent, a hair gel, a hair glaze, a hair dressinger, a hair lacquer, a hair moisturizer, a hair mousse or a hair spray.

9. A health food for preventing and treating alopecia disorder, comprising a norgalanthamine compound of Formula I defined in claim 1 as an active ingredient.

10. The health food according to claim 9, wherein the health food is a powder, a granule, a tablet, a capsule or a beverage.
